(19) United States
(12) Patent Application Publication
(10) Pub. No.: US 2023/0266270 A1
(43) Pub. Date: Aug. 24, 2023

(54) METHODS AND SYSTEMS FOR ANALYZING SAMPLE PROPERTIES USING ELECTROPHORESIS

(71) Applicant: CASE WESTERN RESERVE UNIVERSITY, Cleveland, OH (US)

(72) Inventors: UMUT GURKAN, Shaker Heights, OH (US); MICHAEL HINCZEWSKI, Cleveland, OH (US); SHAMREEN IRAM, Cleveland, OH (US); RAN AN, Shaker Heights, OH (US)

(21) Appl. No.: 18/012,951

(22) PCT Filed: Jun. 28, 2021
(86) PCT No.: PCT/US2021/039385

§ 371 Date: Dec. 26, 2022

Related U.S. Application Data
(60) Provisional application No. 63/044,643, filed on Jun. 26, 2020.

(51) Int. Cl. G01N 27/447; G01N 33/72

(52) U.S. Cl. CPC G01N 27/44721; G01N 33/721; G01N 33/726

(57) ABSTRACT

An example system includes one or more non-transitory machine-readable media and one or more processors. The non-transitory machine-readable media is configured to store data and instructions, in which the data includes image data having a plurality of image frames of an electrophoresis process performed on a sample over a time interval. The sample contains at least one analyte. The one or more processors are configured to access the data and execute the instructions, in which the instructions programmed to perform a method. The method can include determining values of pixels within a region of interest (ROI) of respective image frames in the time interval. The method can also include analyzing the determined pixel values for at least some of the respective image frames. The method can also include estimating a quantity of the at least one analyte in the sample based on the analysis of the pixel values.

### RELATED APPLICATION

This application claims priority from U.S. Provisional Application No. 63/044,643, filed Jun. 26, 2020, the subject matter of which is incorporated herein by reference in its entirety.

### GOVERNMENT FUNDING

This invention was made with government support under Grant Nos. HL140739 awarded by The National Institutes of Health. The government has certain rights to the invention.

### TECHNICAL FIELD

This application relates to systems and methods for analyzing sample properties using electrophoresis.

### BACKGROUND

Anemia affects a third of the world’s population with the heaviest burden borne by women and children. Anemia leads to preventable impaired development in children, as well as high morbidity and early mortality among sufferers. Genetic hemoglobin (Hb) disorders, such as sickle cell disease, are among the major causes of anemia globally. Blood Hb level (in g/dL) is used as the main indicator of anemia, while the presence of Hb variants (e.g., sickle Hb or HbS) in blood is the primary indicator of an inherited disorder. Even though treatments are available for anemia and Hb disorders, screening, early diagnosis, and monitoring are not widely accessible due to technical challenges and cost, especially in low-and-middle-income countries.

Electrophoresis is a technique used to separate particles (or macromolecules) disposed on or in a medium in response to applying an electric field. Electrophoresis may be used to separate molecules based on charge, size and binding affinity. Electrophoresis is often applied to separate and analyze biomolecules, such as DNA, RNA, proteins, nucleic acids, plasmids, and fragments of such biomolecules. More recently, hemoglobin (Hb) electrophoresis has been used for diagnosing blood disorders, such as sickle cell disease and other disorders.

### SUMMARY

One example embodiment includes a system that includes one or more non-transitory machine-readable media and one or more processors. The non-transitory machine-readable media is configured to store data and instructions, in which the data includes image data having a plurality of image frames of an electrophoresis process performed on a sample over a time interval. The sample contains at least one analyte. The one or more processors are configured to access the data and execute the instructions, in which the instructions programmed to perform a method. The method can include determining values of pixels within a region of interest (ROI) of respective image frames in the time interval. The method can also include analyzing the determined pixel values for at least some of the respective image frames. The method can also include estimating a quantity of the at least one analyte in the sample based on the analysis of the pixel values.

Another example embodiment is directed to a method that includes storing image data that includes image frames of an electrophoresis process performed on a sample over a time interval. The method also includes determining values of pixels within a region of interest (ROI) of respective image frames in the time interval. The method also includes analyzing the pixel values for at least some of the respective image frames and estimating a quantity of at least one analyte in the sample based on the analysis of the pixel values. In an example, the method may be executed by one or more processors. In another example, the method can be stored in one or more non-transitory media as machine-readable instructions that are executable by one or more processors.

Yet another example embodiment is directed to a system. An electrophoresis system includes an electrophoresis medium configured to hold a blood sample containing at least one blood analyte and a known calibrator. An imaging system is configured to acquire images of the electrophoresis medium at a frame rate to provide image data having image frames representative of an electrophoresis process performed on the sample over a time interval. One or more non-transitory machine readable media are configured to store data and instructions, in which the data includes the image data. One or more processors configured to access the data and execute the instructions. The instructions include an analyte quantity calculator programmed generate an array having elements that encode image information within a region of interest (ROI) of a plurality of respective image frames acquired during the electrophoresis process. The analyte quantity calculator can be further programmed to apply a machine learning model to analyze the array and provide an indication of a quantity of the at least one blood analyte in the sample.

Yet another example embodiment is directed to a computer-implemented method. The method includes generating an array having elements that encode image information within a region of interest (ROI) of a plurality of respective image frames acquired during an electrophoresis process performed on a blood sample. The method also includes applying a machine learning model to analyze the array to provide an indication of a quantity of at least one blood analyte in the blood sample.

### BRIEF DESCRIPTION OF THE DRAWINGS

FIG. 1 is a block diagram of a diagnostic system for determining a quantity and/or variant(s) of an analyte within a sample.

FIG. 2 is a block diagram depicting an example preprocessing function that can be applied image data.

FIG. 3 is a block diagram depicting an example of an analyte quantity calculator.

FIG. 4 is a block diagram depicting an example of an analyte variant calculator.

FIG. 5 is a flow diagram depicting an example method for determining a quantity and/or one or more variants of an analyte within a sample under test.

FIG. 6 depicts a schematic example of a diagnostic process that can be implemented to characterize one or more analytes in a sample.

FIGS. 7A-7F depict an example of an overall system workflow for characterizing the features of an analyte within a sample medium.

FIGS. 8A-8T depict example outputs of an integrated diagnostic system for characterizing different analytes using electrophoresis.

FIGS. 9A and 9B show examples of hemoglobin levels for different samples.

FIGS. 10A-10C are plots showing example results of predicted hemoglobin values relative to hemoglobin levels determined according to other methods.

### DETAILED DESCRIPTION

This disclosure provides systems and methods to analyzing and characterizing sample properties using electrophoresis.

In an example, systems and methods can use an imaging modality (e.g., a digital camera) to record electrophoresis performed on a sample containing one or more analytes. During electrophoresis, for example, different (bio)molecules (e.g., including total hemoglobin, a standard calibrator, and hemoglobin variants) can be separated based on their charge-to-mass ratio in response to an electric field in the presence of a carrier substrate (or other electrophoresis medium). Image frames for the process can be generated and stored in memory. Image processing (e.g., computer vision) can analyze the image frames, such as to identify and track band position and/or movement within a region of interest (ROI). The ROI can be defined according to the electrophoresis system and imaging system being used to record the electrophoresis process in a reproducible manner. Computer vision then processes the image frames (e.g., and generates an array for each respective image frame. For example, the array is generated as a time series vector, which can encode the relative information between the analyte band(s) and a standard calibrator band. The array (e.g., a time series vector) can provide an input to a trained machine learning model. The trained machine learning model is programmed to perform pattern recognition and regression analysis by examining underlying input data, such as is encoded using the time array, to predict the analyte level. In some examples, the model can also be trained to detect or diagnose a condition or disorder based on the estimated analyte level. As described herein, the systems and methods can also be configured to perform analyte variant identification based on analysis of respective image frames acquired during the electrophoresis process.

In some examples, the systems and methods can use a trained machine learning model to analyze and recognize the underlying pattern of the relative Hb intensity and associate this pattern with a hemoglobin level and corresponding anemia status. Within the same test, a variant calculator can analyze Hb variant band migration and identify one or more Hb variants. The systems and methods disclosed herein allow accurate, reproducible blood Hb level prediction and anemia detection in paper-based Hb electrophoresis, which is a clinical standard test for Hb variant screening and diagnosis worldwide.

While many examples herein describe systems and methods to analyze images of electrophoresis to determine properties of blood samples, such as Hb or serum protein levels as well as to identify Hb variants, the systems and methods described herein are equally applicable to analyze electrophoresis applied to other types of blood analytes as well as other types of samples, which may include biological or non-biological samples. For example, electrophoresis is a very broadly used technique which, fundamentally, applies electric current to molecular structures. The separation that occurs in response to applying electrophoresis to the sample depends on the mass and charge of one or more respective analytes in the sample.

As used herein, in the context of electrophoresis, the term analyte and its variants refer to one or more physical components (e.g., substances or materials) capable of being analyzed using electrophoresis. For example, an analyte can be naturally occurring within a sample or it can be added to the sample, directly or indirectly, for purposes of analysis. In some cases, the sample can itself be the analyte. Examples of analytes can include proteins (e.g., hemoglobin), chemical compounds, nucleic acids, DNA, RNA or other molecules or structures having properties of both mass and charge.

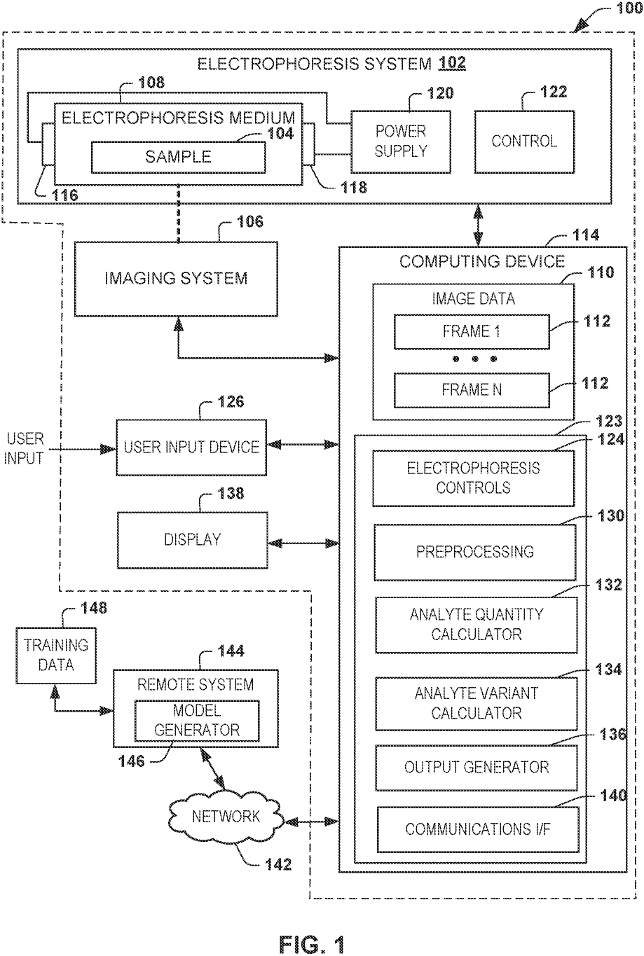

FIG. 1 depicts an example of a diagnostic system 100 that can be implemented to characterize one or more analytes contained within a sample on which electrophoresis is performed. The diagnostic system 100 includes an electrophoresis system 102 configured to perform electrophoresis on a sample 104. An imaging system 106 is configured to record images of an electrophoresis medium 108 during a test time interval to provide corresponding image data 110 that includes a plurality of image frames 112 shown as frames 1 — N, where N is a positive integer denoting the number of image frames. The number of frames N can vary depending upon the length of the test interval and the frame rate at which the imaging system 106 records the image frames. The image data 110 can be stored in one or more non-transitory machine-readable media (i.e., memory) of a computing device 114. The memory can include volatile and non-volatile memory, and may be local to the system, external to the system or be distributed memory. The image data 110 can also be stored in the imaging system 106 and then transferred to corresponding memory of the computing device 114 during or after the test interval has completed.

The electrophoresis medium 108 is configured to carry or hold the sample 104. The electrophoresis medium 108 can be coupled between respective electrodes 116 and 118 (also referred to as terminals). A power supply 120 has outputs coupled to the respective electrodes 116 and 118. The power supply 120 is configured to provide a voltage potential across the electrodes 116 and 118 to generate an electric field across the electrophoresis medium that contains the sample 104. The power supply can be implemented as a switch mode power supply, transformer or a combination of power supplies configured to supply a voltage across the electrodes 116 and 118, a control circuit 122 is coupled to the power supply 120 and configured to control the power supply for delivery of the voltage potential across the respective electrodes 116 and 118.

For example, the control circuit 122 can be configured to detect the presence of the electrophoresis medium 108 within the electrophoresis system and to activate the power supply in response to detecting its presence. Alternatively, or additionally, the control circuit 122 can be configured to control the power supply 120 in response to a user input that is provided to activate the electrophoresis process. The user input can be provided by a user input device (e.g., device 126) or an associated computing device 114.

As an example, a sample 104 can be prepared by diluting the sample into a calibrator solution. The mixed sample is then loaded into the electrophoresis medium 108 (e.g., wetted cellulose acetate paper). A volume of background electrolyte can also be injected into buffer ports at each end of the cartridge containing the medium 108 to provide sufficient contact between electrode-electrolyte-medium to complete circuit connection and provide stable current for performing electrophoresis. A buffer solution can also be introduced and applied to the electrophoresis medium, such as to configure a pH for the sample to enable or facilitate electrophoresis for different types of molecular structures. In one example, when the sample 104 includes blood, a Tris/Borate/EDTA (TBE) buffer is used to provide ions to enable electrical conductivity at a pH of about 8.4 in the electrophoresis medium 108. The pH induced net negative charges of the hemoglobin and the standard calibrator molecules cause them to travel from the negative to the positive electrode in response to an applied electric field between respective electrodes 116 and 118.

The electrophoresis system 102 thus can be configured to perform electrophoresis on the electrophoresis medium 108 containing the sample 104. The medium 108 can vary depending upon the type of electrophoresis being implemented. For example, the electrophoresis medium 108 can be a cellulose paper, a gel or other forms of media configured to provide an electric field across the medium and a sample contained there within. The components disposed within the sample (e.g., analytes and/or calibrator) exhibit electrophoretic propagation thereof across the medium in response to the applied electric field between the terminals 116 and 118. The electrophoretic propagation of components varies depending upon the charge-to-mass ratio of the analyte, calibrator as well as other components within the sample 104 responsive to the electric field that is provided by the power supply 120.

The type of analyte (or analytes) can vary depending upon the sample under test. Examples of samples can include an immunoassay, a nasal swab, sputum, other biological samples, as well as non-biological materials (e.g., bodily fluids, gun powder, natural or synthetic chemical compounds, organic materials, inorganic materials, and the like). As one example, the sample 104 can include blood and the analyte includes hemoglobin or serum proteins. For example, plasma can be separated from the blood and the plasma be inserted in the electrophoresis system as a sample 104, and the analyte can include one or more serum proteins (e.g., albumin, globulins, fibrinogen and the like).

As one example, the diagnostic system 100, including the electrophoresis system 102, can be implemented according to the example embodiments in U.S. Pat. No. 10,375,909, which is incorporated herein by reference. As another example, the diagnostic system 100 can include an electrophoresis system 102 implemented according to the disclosure in U.S. Pat. Publication No. 2017/0227495, which is incorporated herein by reference. As a further example, the medium 108 can be implemented as the HemeChip cartridge is composed of two injection molded plastic parts made of Optix® CA-41 Polymethyl Methacrylate Acrylic, such as is commercially available from Hemex Health® of Portland, Oregon. For example, the injection molded HemeChip cartridge embodies a pair of electrodes 116 and 118, which can be formed of corrosion-resistant, biomedical grade stainless steel. HemeChip electrodes provide oxidative resistance, stability against electrochemical reactions during operation, and reliability of electrical connection with the power supply 120. For example, the combination of high stability cellulose acetate paper used as the medium 108, injection molded Polymethyl Methacrylate Acrylic plastic, and corrosion-resistant biomedical grade stainless-steel electrodes 116 and 118 help to improve longevity. While many of the following examples relate to a cellulose paper electrophoresis medium 108, such as implemented in the HemeChip cartridge, the systems and methods described herein are equally applicable to other types of electrophoresis test systems (e.g., using capillary-based, gel-based or film-based electrophoresis).

In some examples, the imaging system 106 (e.g., including optics and a digital camera) can be integrated in a housing with the electrophoresis system 102 and be configured to record respective image frames (e.g., digital image frames) of the electrophoresis process on the sample 104 is contained within the medium 108. In another example, the imaging system 106 can be a remote digital camera having a field of view that includes the electrophoresis medium and/or another image of the electrophoresis medium such as may be provided on a display or visible through a window of the electrophoresis system 102. The imaging system 106 provides image data 110 that includes a plurality of image frames 112 based on a frame rate at which the frames are acquired by the imaging system. The frame rate may be fixed or it may be variable. In one example, the frame rate may be one frame every ten seconds or faster, such as one frame every seven seconds, one frame every 4 seconds, one frame every two seconds, one frame per second or up to a rate of full motion video (e.g., about 16 frames per second). The number of frames determine the resolution of the electrophoresis information contained in the image data 110.

The computing device 114 can be programmed with instructions 123 that include electrophoresis controls 124. The electrophoresis controls 124 can be programmed to provide instructions to the control circuitry 122 for controlling operating parameters of the electrophoresis system 102. For example, a user input device 126 can be coupled to or part of the computing device 114 and, in response to a user input via the user input device 126, can operate the electrophoresis controls 124 to activate or deactivate the electrophoresis system 102. As an example, the user input device 126 can be implemented as a keyboard, mouse, touch screen interface, or a remote user device (e.g., a cellphone or tablet connected to the computing device through an interface) to enable user interaction with the computing device 114 as well as the diagnostic system 100, more generally.

The computing device 114 also can be coupled to the electrophoresis system 102, including the control circuitry 122, such as a physical connection (e.g., electrically conductive wire, optical fibers) or a wireless connection (e.g., WiFi, Bluetooth, near field communication, or the like). In one example, the computing device 114 can be integrated in the housing of the system 100 with the electrophoresis system 102. Alternatively, the computing device 114 can be external to the housing, which contains the electrophoresis system 102, and be coupled to the electrophoresis system 102 via a physical and/or wireless link.

The computing device 114 includes instructions (e.g., a set of program code modules) 123 executed by one or more processors of the computing device 114 to perform various methods and functions disclosed herein. As described herein, the instructions 123 are configured to analyze the sample 104 based on the image data 110 such as to characterize properties of the sample 104, including to identify the presence or absence of and/or one or more analytes within the sample. The instructions can also be configured to quantify (e.g., provide a measure, such as concentration) of analytes that have been identified. The computing device 114 can also be configured to determine a condition (e.g., a diagnosis) of the subject providing a biological sample based on the image data 110.

In an example, the computing device 114 includes a preprocessing function 130 to process the image data into a form to facilitate processing and analysis. For example, the preprocessing function can be programmed to define a region of interest in each of the respective image frames 112. The region of interest can be predefined such as by providing coordinates or boundaries of the region within the field of view of the imaging system 106. The region of interest can include two-dimensional array of pixels in each of the respective image frames. In another example, the region of interest can be a single line of pixels across the field of view (e.g., a longitudinal set of pixels on a surface of the medium extending between electrodes 116 and 118). The preprocessing function 130 can extract the region of interest from each of the respective image frames. Alternatively, the imaging system 106 can be configured to generate the respective image frames 112 to include only the pixels from the predefined region of interest. In some examples, the region of interests can be programmable in response to the user input using the corresponding user input device 126.

As a further example, the preprocessing function 130 can also include a pixel scaling function. For example, the pixel scaling function 130 is programmed to scale pixels in each of the respective image frames so that pixel values within each respective image frame are normalized for a respective electrophoresis process. As used herein, each pixel value can include an intensity value. For example, each pixel value includes an intensity value within a range of values established for one or more color channels, which can vary depending on a color model being used. A pixel value can also include spatial coordinates of the respective pixel in a respective image frame and/or time (e.g., absolute and/or relative time). In one example, the preprocessing function 130 is programmed to employ a threshold operator to each of the respective color channels in the image data 110. The threshold operator can scale pixel values for each of the color channels (e.g., according to a color model, such as any of the RGB, YUV or YCbCr color models) in each respective frame 112. The threshold operator can also remove white and black pixel values from each of the respective color channels. Thus, the preprocessing function can generate scaled image data in which each of the color channels for respective pixels in each of the respective image frames 112 are normalized, such as on frame-by-frame basis or across the set of image frames. For example the scaling can adjust the values associated with analyte in one color channel and adjust the values for a calibrator in the sample in another color channel. That is, the preprocessing function is configured to determine values of pixels within a region of interest of the respective image frames 112 in a time interval in which the electrophoresis process is implemented for a given sample 104.

The instructions 123 executable by the one or more processors of the computing device 114 also include an analyte quantity calculator 132. The analyte to quality calculator 132 is programed to analyze the determined pixel values for some or all of the respective image frames 112 and estimate properties of one or more analytes in the sample 104 based on such analysis. The estimated properties can include an identification of one or more analytes present or absent from the sample as well as indicate a quantity of one or more identified analytes. The quantity can be a binary value, such as to indicate the presence or absence of a respective analyte, or it can be a value specifying a level (e.g., concentration) thereof.

In one example, the analyte quantity calculator 132 is programmed to determine a relative pixel value for each of the image frames 112 based on values of pixels in a first color channel associated with an analyte and evaluate pixels in another color channel associated a calibrator. The analyte quantity calculator 132 can generate a frame array data structure having respective data elements that are determined based on pixel values for each respective frame. In one example, the frame array is implemented as a vector (e.g., one-dimensional time-series vector ρ(t)) of the relative pixel values (e.g., pixel intensity) determined for each of the respective frames 112. In another example, the analyte quantity calculator 132 can implement the frame array as a multidimensional array configured to encode the information in a respective image frame 112 using more than one value (e.g., encoding, pixel values for respective channels, spatial information across the region of interests and/or temporal information). In yet another example, each of the elements of the array can include the entire image frame 112, such as including the scaled pixel values for the ROI that has been determined for each of respective frames 112.

In some examples, the analyte quantity calculator 132 includes a machine learning model that is trained to recognize patterns in the respective image frames 112 (or in data derived from the image frames) to classify the patterns based on predetermined features in respective image frames, which features correlate to properties of one or more analytes, and to predict a quantity and/or other property of the analyte or analytes in the sample 104. For example, the machine learning model implemented by the analyte quantity calculator 132 is programmed to perform pattern recognition and regression analysis to predict an indication of the quantity of the analyte in a sample. The machine learning model implemented by the calculator 132 can utilize one or more types of models, including support vector machines, regression models, self-organized maps, k-nearest neighbor classification or regression, fuzzy logic systems, data fusion processes, boosting and bagging methods, rule-based systems, artificial neural networks or convolutional neural networks. When image processing is applied to encode the respective image frames, the training and resulting ML model can be efficiently implemented, reducing computational and memory storage requirements.

In one example, when the analyte includes Hb, the machine learning model can be implemented as an artificial neural network (ANN) trained to evaluate the relative intensity between Hb band and the standard calibrator band. The trained ANN can thus be programmed to perform pattern recognition and regression analysis by examining underlying input data, which is encoded using the time series vector ρ(t), to predict an estimate of the Hb level (e.g., in g/dL or another unit of measure). The ANN (or other function implemented by the analyte quantity calculator 132) can also diagnose a blood disorder based on the estimated Hb level, such as anemia or other blood condition. For example, if a patient’s age and/or sex is known, the predicted Hb level can be compared to one or more thresholds (e.g., implemented in a look-up table) to provide the diagnosis of anemia.

The instructions 123 executable by the one or more processors of the computing device 114 can also include an analyte variant calculator 134. The analyte variant calculator 134 is programmed to identify one or more analyte variants based on analysis of the pixel values in the respective image frames 112. In an example, the analyte variant calculator 134 can analyze a subset of the respective image frames 112, such as corresponding to a later portion of the electrophoresis process. In another example the analyte variant calculator 134 can determine the one or more analyte variants based on analysis of the complete set of image frames 112.

In an example when an analyte in the sample 104 is hemoglobin, the analyte variant calculator is programmed to analyze the respective pixel values in respective image frames 112 to identify at least one hemoglobin variant for the sample 104 based on a distribution of Hb electrophoretic bands during and/or after separation shown in one or more image frames. For example, the hemoglobin variant can be of hemoglobin phenotype collected from HBAA, HBSA, HBSS, HBSC and HBA2. In one example, the analyte variant calculator 134 is programmed to employ algorithms to determine one or more variants of the analyte. As one example, the analyte variant calculator can be programmed to implement the analyte categorization method disclosed in Hasan, M.N., A. Fraiwan, et al., Paper-based microchip electrophoresis for point-of-care hemoglobin testing. Analyst, 2020; 145(7): p. 2525-2542.

In other examples, the analyte variant calculator 134 includes a trained deep machine learning model that can be applied to respective image frames 112 to determine one or more variants of the analyte in the sample 104. For example, the machine learning model can be implemented as a convolutional neural network (CNN) having a plurality of layers trained to analyze some or all of the respective image frames 112 (e.g., having scaled pixel values) to perform corresponding pattern recognition and regression analysis to identify one or more variants of the analyte in the sample. In another example, the CNN can be trained to analyze a frame array (e.g., generated to encode respective image frames 112) and perform corresponding pattern recognition and regression analysis to identify one or more variants of the analyte in the sample 104.

The patterns can include patterns of pixels in a single frame, patterns of pixels across multiple frames (e.g., over time) or patterns from the full set of image frames acquired during the electrophoresis process. Features can include pixel values, such as the intensity of bands (actual or relative intensity) and/or distribution of bands in respective image frames. In an example, the training data can include sets of electrophoresis image data acquired for an ROI for samples containing known analytes having known properties. For example, a study can be designed to acquire image data that includes blood samples from patient populations having a broad range of Hb levels (e.g., low Hb levels, medium Hb levels and high Hb levels). The Hb levels can be determining using standard measures, such as actual complete blood count (CBC) reported results for each of the blood samples.

The instructions 123 implemented by one or more processors of the computing device 114 can also include an output generator 136. The output generator 136 is programmed to generate the output data that can be provided to a display device 138 or other output device to provide a tangible representation of the analyte quantity and/or analyte variant that has been determined by calculator 132 and/or 134. For example, the output generator 136 can provide a textual and/or graphical representation to the display 138 specifying a level of the analyte within the sample 104. For an example of a hemoglobin analyte, the output generator 136 can be configured to specify blood HD level in g/dl. The output generator 136 can also specify one or more variants of the hemoglobin or other analytes being detected in the sample 104 as well as a respective percentage of the variant(s). The output generator 136 can also generate output data that includes an indication of an absence or indetectable quantity of the analyte or component of interest.

The computing device 114 can also include a communications interface 140 to communicate through one or more networks 142, such as for communications with a remote system 144. The communication interface 140 can be implemented to communicate with the network 142 using one or more physical connections (e.g., an electrically conductive connection or optical fiber), one or more wireless links (e.g., implemented according to an 802.11x standard or other short-range wireless communication) or a network infrastructure that includes one or more physical and/or wireless communications links.

The remote system 144 can include a server, a general purpose computing device (e.g., notebook computer, laptop, desktop computer, workstation, smartphone or the like) and/or it can be a special purpose system configured to interact with one or more of the diagnostic systems 100 via the network 142. In another example, the remote system 144 may send program instructions to the computing device 114 to configure and/or update its operating program instructions 123. The remote system 144 can include a model generator 146 that is programmed to execute instructions for generating one or more machine learning models that can be provided to the computing device 114 to the network 142. For example, the model generator 146 is configured to generate the one or more models based on the respective training data 148, such as described herein.

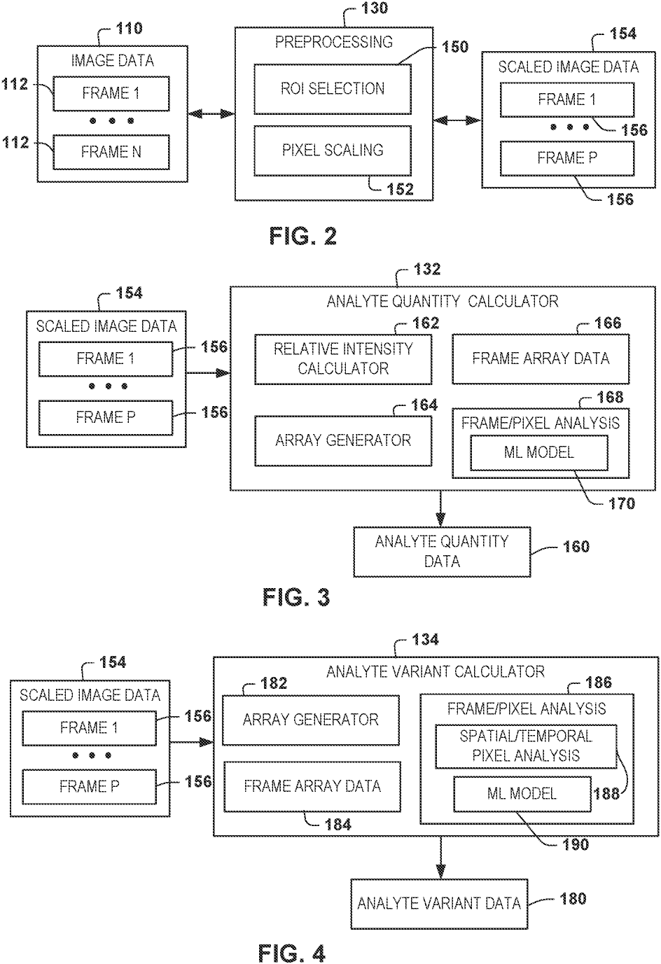

FIG. 2 is a block diagram depicting an example of the preprocessing function 130. The preprocessing function 130 is programmed to perform image processing with respect to the image data 110 including with respect to each of the respective image frames 112 acquired by the imaging system during electrophoresis process (e.g., implemented by electrophoresis system 102). The preprocessing function 130 includes an ROI selection function 150 programmed to define the ROI that is used in each of the respective image frames. As an example, the ROI selection function 150 can define pixel coordinates in the image frame to define which pixels (e.g., a one- or two-dimensional array of pixels) are to be further processed and which pixels may be deleted or discarded from the image data 110. Because different imaging systems and sets may be used to acquire the image data 110, the ROI selection function 150 may utilize different pixel coordinates to set the ROI depending upon the set up and/or imaging system 106. For example, the aspect ratio of the ROI may remain fixed throughout the different potential setups but shifted to avoid boundaries and other data that may be outside of the desired ROI.

Preprocessing function 130 also includes a pixel scaling function 152. The pixel scaling function 152 can be programmed to scale or normalize values of pixels across each of the respective color channels utilized in the image data 110. As an example, the imaging system (e.g., imaging system 106) can utilize a red, green, blue (R, G, B) color model including respective color channels having pixel values (e.g., bit values ranging from 0 to 255). In other examples, respective pixels may be assigned values according another color model, such as the YUV or YCbCr color model. In one example, the red color channels is used to identify pixels associated with a given analyte in the sample, and the blue color channel is utilized to represent a known calibrator. In the red color channel, a scaling function can be applied to each pixel to normalize the red pixel values with respect to green and blue pixel values in respective color channels. For example, a scaled red value (Rs) for a given pixel can be expressed as Rs = R-½ (G+B), where R is the value of a given pixel in the red channel, G is the value of the given pixel in the green channel and B is the value of the given pixel in the blue channel. Similarly, a blue scaled (B_{S}) value for a given pixel may be expressed as by: B_{S} = B-½ (R+G). By implementing the above functions, the pixel scaling function 152 also maps all white pixels from the channels to 0, to facilitate the removal from the image data by preprocessing function 130. Additionally, all black pixels mapped to a high pixel value (e.g., 255). In other examples, different numbers and types of color channels may be implemented having different ranges of pixel values for each color channel for each of the respective pixels. The processing function 130 in turn generates scaled image data 154 that includes a plurality of frames 156, shown as frames 1 - P, where P is a positive integer denoting the number of frames in the scaled image data 154. In some examples P = N so that the number of frames in the original image data 110 is the same as the scaled image data. In other examples, P can be less than N such that some frames may be omitted from the original image data 110 for further processing by the instructions 123 implemented by the computing device 114.

FIG. 3 is a block diagram depicting an example of the analyte quantity calculator 132 that may be implemented by the computing device 114 to determine analyte quantity data 160. In an example, the analyte quantity data 160 can specify a level of a given analyte within a sample based on analysis of the imaging data, such as the scaled image data 154. In some examples, the preprocessing function 130 may be omitted so that the analyte quantity calculator 132 determines the analyte quantity data based on the original (not scaled) image data 110. The analyte quantity data 160 can also specify a condition based on the determined level, such as a diagnosis for biological samples.

For example, the calculator 132 includes a relative intensity calculator 162 configured to determine a relative intensity of respective color channels for each pixels of each of the respective image frames 156. As an example, the relative intensity calculator 162 is programmed to sum all pixel values in the ROI of a given frame for the first color channel and all pixel channels in the ROI in the given frame for a second color channel. For example, the pixel values can correspond to the scaled pixel values determined by the processing function 130. The relative intensity pᵢ can be determined as the ratio of the sum of pixel values for the analyte (corresponding to a first color channel) divided by the sum of pixel values for a known calibrator (corresponding to a second color channel) such as can be expressed for a respective image frame, as follows:

$$\begin{matrix}{p}_{{}_{i}} = ( \sum ( \text{pixel values\_channel\_1)/(} \sum \text{pixel values\_channel\_2)} \\ \text{where i denotes a given frame (i=1 to N; or i=1 to P)}\end{matrix}$$

The relative intensity calculator 162 thus can compute a relative intensity value pᵢ for each of the respective frames 156.

The analyte quantity calculator 132 can also include an array generator 164 programmed to generate frame array data 166. For example, an array generator 164 is programmed to generate the frame array 166 as a time-based frame array having elements representative of information in the ROI of the respective image frame for at least a portion of such frames. In one example, the array generator 164 generates the time-based array 166 in which each of the elements of the array include the relative intensity values pᵢ for each of the respective frames. In this way, the frame array data 166 can encode the relative intensity information for the respective frames based on the proportion of the pixel values in the color channel that is representative of the analyte relative to the pixel value representative of the calibrator over time. That is, the frame array data 166 can correspond to signatures encoded in the relative intensity spectrum obtained from the electrophoresis process as contained in the frames 156 of scaled image data 154. Where the elements of the frame array data 166 are relative intensity values pᵢ the array can be stored at a one-dimensional array (e.g., a time-series vector), which encodes the relative intensity information during the test interval according to the set of frames included in the image data 154.

In one example, the array generator 164 can be programmed to generate the frame array data to include relative intensity values for only a proper subset of the frames 156 such as to include a first portion (e.g., frames acquired during a first-time interval of about 100 to 200 seconds, such as 150 seconds). In another example, the array generator 164 generates the frame array data to encode the information in the respective frames during the entire test interval (e.g., of about up to 400 seconds). While the example noted above generates the frame array 166 to include relative intensity values as array elements, in other examples the array generator 160 can be programmed to determine (e.g., extract) additional information from the image frames 156, such as multi-dimensional features extracted from each of the respective image frames. For example, in addition to pixel values in the ROI of each of the respective frames, spatial coordinates of each pixel may also be included in the frame array. As another example, the array generator 164 can include each of the pixels and coordinates in the ROI for each respective frames includes actual image data acquire and preprocessed.

The analyte quantity calculator 132 includes a frame/pixel analysis function 168 programmed to estimate a quantity of one or more analytes in the sample based on the analysis of the pixel values in the image data 154. As an example, the analysis function 168 can estimate the quantity of the analyte based on the encoded image data stored in the frame array data 166 for the respective image frames. As described above, the information in the frame array data may encode the relative pixel intensity between the analyte and calibrator for each image frame. In another example, the frame array can encode other image features that can be extracted from the respective image frames 156.

As a further example, the analysis function 168 can include a trained machine learning (ML) model 170. The analysis function can apply the ML model 170 to the elements of the frame array data for some or all of the respective P frames 156 to determine the analyte quantity data that represents a level of the analyte in the sample under test 104. For example, the ML model 170 can be applied to frame and array elements that encode a proper subset of the image frames (e.g., less than P elements). In another example the ML model 170 may be applied to elements in the frame array data 166 that have been generated for each of the P frames (e.g., where P = N) to determine the analyte quantity data 160. As described herein, the sample can be blood and the analyte can be hemoglobin or another blood analyte. Thus the level of hemoglobin can be provided as part of the analyte quantity data 160 and specify the units in g/dL. The ML model 170 can be trained to classify a condition of the analyte (e.g., anemia for a blood sample) based on the analyte quantity. As described herein, the ML model 170 can be trained to quantify the analyte and determine a condition thereof based on the frame information that has been encoded in the elements of the frame array data over the test interval. By implementing the frame array data as a one-dimensional vector, the machine learning model can be simple and programmed in the computing device 114 including when implemented as a portable apparatus such as a point-of-care device. Additionally, the analyte quantity calculator 132 can be programmed to recognize and quantify more than one analyte that may be present, indetectable, or absent in the sample under test.

By way of example, the ML model 170 can be trained to generate an ANN based on a training data set (e.g., training data 148) having known analyte properties. For example, a processing pipeline was set up using the open source Keras machine learning library on top of a TensorFlow backend. For choice of ANN performing this regression problem, a vanilla feed forward network can be used. Alternatively or additionally, a multilayer perceptron (MLP) can be used for training the ANN. In an example, the constructed ANN has three densely connected layers: an input layer, a hidden dense layer, and an output layer. The input and hidden layers each have 32 nodes with rectified linear unit (ReLU) activations. The ANN takes the pre-processed relative intensity ratio time series vector ρ(t) as input feature vector. The input vector ρ(t), the input feature vector can include thousands (e.g., about 7000 or more) of trainable parameters in the neural network.

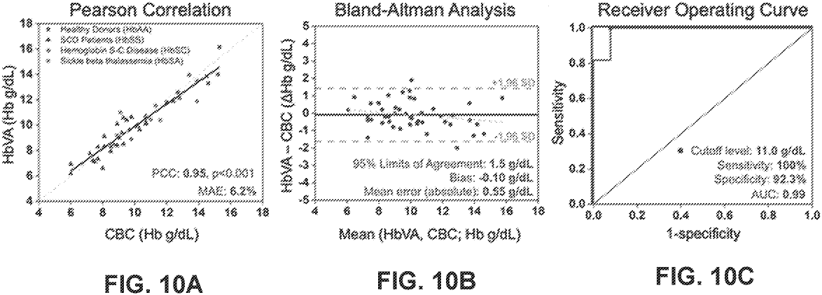

As a further example, the network was trained and tested on a comprehensive data set of 68 tests. The training set consisted of 27 samples, out of which 4 were further split into a validation set (e.g., 15% of training set). The remaining 41 samples were kept aside for the test set, and later augmented by a further set of 5 samples to make up a combined test set of 46 samples. Training was run on an NVIDIA GeForce RTX™ 2060 GPU. Assuming the error in the input data CBC responses to be normally distributed, the mean squared logarithmic error (MSLE) can be selected as a loss function to minimize over the training process. Other loss functions could be used in other examples. To prevent overfitting, along with allocating 15% of our training set into a holdout validation set, training was stopped when the validation loss performance stopped changing over a set number of epochs. The optimal network reached an MSLE loss of 0.9% for training and 1.1% for validation (see, e.g., FIGS. 10A-10C for examples of results and validation metrics.

FIG. 4 depicts an example of an analyte variant calculator 134 programmed to generate analyte variant data 180. For example, the analyte variant data 180 can specify one or more variants of respective analytes that may be present, indetectable, or absent in the sample under test (e.g., sample 104) during an electrophoresis process. Thus the analyte variant calculator 134 is programmed to analyze the image data, such as the scaled image data 154, which includes respective image frames 156 to ascertain variants of the analyte. In the example when the analyte is hemoglobin, the analyte variant data can identify one or more hemoglobin variants in the sample as well as the relative percentage of the variants that constitute the hemoglobin analyte in the sample. Examples of hemoglobin variants include HbAA, HbSA, HbSS, HbSC, and/or HbA2, each of which exhibits different relative separation along the medium during electrophoresis.

In some examples, the calculator 134 includes an array generator 182 that is programmed to construct an array based on features contained in the respective image frames. Each of the image frames or selected portion of the P image frames can be used by the array generator 182 in constructing frame array data 184. For example, the array generator can include a feature extractor programmed to process each of the respective image frames to generate a corresponding feature set that can be stored as respective elements from the array 184 for encoding each of the respective image frames. The features can encode individual spatial and/or temporal information (e.g., pixels or aggregate pixels distributed throughout the ROI) in each of the respective image frames. As described herein, the frame array data 184 can also include each respective entire image frame 156 (or 112), such that the frame array data includes an array in which each of its elements is a respective image frame to define corresponding time-series data.

Variant calculator 134 also includes a frame/pixel analysis function 186. The analysis function 186 is programmed to analyze the frame array data 184 to generate the analyte variant data 180. This type of analysis implemented by the analysis function 186 can vary depending upon the features and information included in the elements in the frame array data 184. In one example, spatial/temporal pixel analysis function 188 is programmed to analyze spatial distribution and pixel values provided as elements in the frame array data 184 to identify analyte variants in the data 180, such as disclosed in Hasan, M.N., A. Fraiwan, et al., Paper-based microchip electrophoresis for point-of-care hemoglobin testing. Analyst, 2020; 145(7): p. 2525-2542.

In another example, the analysis function 186 is programmed to include a machine learning model 190 that is trained to classify the contents of the frame array data 184 into one or more variants of a given analyte or multiple analytes. For example, the machine learning model 190 can be implemented as a CNN that is trained to process the elements and features in the frame array data 184 to classify respective pixels and cluster of pixels as they separate and move throughout the ROI of the respective image frames. The ML model 190 thus can distinguish and classify the variants of a given analyte as well as estimate the relative percentage of each variant in the sample.

As one example, the preprocessing function 130 is further programmed to extract the ROI from each frame of the image data (e.g., acquired as video). The preprocessing function 130 can normalize respective pixels in the ROI the function described above (e.g., to isolate red and blue components against a white or gray background). The stack of ROIs over time can form a 3D image, in which each pixel is specified by: (normalized value, horizontal location of pixel, vertical location of pixel, time). Alternatively preprocessing function 130 can be programmed to compress the frame data 156 to a 2D image. For example, the preprocessing function 130 can sum over the vertical locations at each horizontal position, in which case each pixel in the 2D image be specified by: (horizontal location, time). The resulting image data is provided as an input to the machine learning model 190.

As a further example, when 3D image are used as the input and the model 190 is implemented as a CNN, the CNN will be a 3D CNN. The 3D CNN can include blocks, in which each block consists of the following types of layers: 3D convolution layers, 3D max pooling layers, batch normalization layers and dropout layers. Several of these blocks will be used consecutively (e.g., the optimal number of layers to be determined based on the particular application), followed by one or two dense layers and finally softmax, which provides an output node for each respective output category (e.g., the type of analyte variant). In another example, when the 2D image data is used as the input, a similar network architecture applies, except that 2D convolutional and max pooling would be used instead of respective 3D versions.

In an example, the CNN may be adapted from a deep learning, residual neural network, such as ResNet-50, and may be pretrained on ImageNet. The CNN can be trained based on samples having known analyte variants (e.g., known Hb variants), such as including a set of discrete variants (e.g., HbAA, HbSA, HbSS, HbSC, and/or HbA2) or variants could be categorized over a continuous range. For example, a subset of the patient samples will be taken for training, with each sample labeled by the variant associated with the respective patient. The CNN will learn to associate the patient data (e.g., 3D or 2D image data) with the variant label. The network can then be tested on a subset of the samples, such as according to a k-fold cross-validation protocol to validate the accuracy of our machine learning model 190. While the above example, describes the ML model 190 as being a CNN, other types of machine learning models may be implemented in other examples.

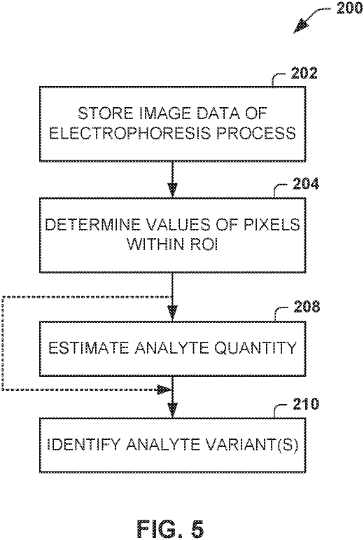

FIG. 5 depicting an example of a method 200 for analyzing image data representative of an electrophoresis process performed on a sample. While for purposes of simplicity of explanation, the example method 200 of FIG. 5 is shown and described as executing serially, the example method is not limited by the illustrated order, as some actions could in other examples occur in different orders, multiple times and/or concurrently from that shown and described herein. Additionally, the method 200 can be implemented as machine-readable instructions executed by a processor, such as by the computing system 114 of FIG. 1, including functions and methods of FIGS. 2-4. Accordingly, the description of FIG. 5 also may refer to FIGS. 1-4.

At 202, the method includes storing image data that includes image frames of an electrophoresis process performed on a sample over a time interval. As described herein, an electrophoresis medium (e.g., medium 108, such as a gel or cellulose paper medium) can contain the sample can during the electrophoresis process in which an electric field is applied across the medium to cause electrophoretic movement of one or analytes and a calibrator. In this way each of the image frames includes pixels that describe a position and concentration of electrophoretic bands as they traverse the medium. In one example, the sample is a blood sample and the at least one analyte is a blood analyte, such as Hb or serum protein. Other types of samples and corresponding analytes can be used in other examples.

At 204, values of pixels within a region of interest (ROI) of respective image frames in the time interval are determined. In one example, the respective image frames are multi-channel images having multiple color channels. The values of pixels in such multi-channel example can be determined at 204 by scaling value of each pixel for a respective color channel with respect to values of the pixel for other color channels in each of the respective image frames. Resulting scaled values for the pixels in each the respective image frames can be provided and stored in memory (e.g., as scaled image data 154) for further processing at 208-210.

In a further example, the scaling of pixel can include normalizing a value of each pixel in a first color channel, which represent pixels corresponding to the at least one analyte in the sample, based on a combination of its value in the first color channel as well as one or more values of each such pixel in other color channels. Additionally, the scaling can also normalize a value of each pixel in a second color channel, which represent pixels corresponding to a calibrator in the sample, based on the value of the respective pixel in the second color channel and its values of each respective pixel in other color channels, including the first channel.

At 208, a quantity of at least one predetermined analyte in the sample is estimated based on the values of the pixel values (determined at 204). When the sample is a blood sample, the quantity of the at least one analyte can include an indication of blood hemoglobin level in the sample. The method can also provide a diagnosis of anemia based on the indication of blood hemoglobin level in the sample. Alternatively, the quantity of the at least one analyte comprises an indication of serum protein level in the sample.

As a further example, the determination of analyte quantity at 208 can include further analysis of the image frames (e.g., frames 112 and/or 156). For example, a frame array can be generated to have elements that encode information in each respective image frame for a portion (up to the entirety) of the electrophoresis time interval. In an example when the respective image frames are multi-channel images, a first color channel can represent values of pixels corresponding to the analyte in the sample and a second color channel can represent values of pixels corresponding to a calibrator. In such example, each frame element can be determined based on a relative pixel value for each of the image frames, such as a function of pixel values in the first and second color channels and values of pixels in the second color channel for the respective image frame. In this way, the frame array (e.g., data 166) can provide a vector that includes values of the relative pixel intensity for one or more color channels that encodes information in the respective image frames.

The analyte quantity in the sample thus can be determined (at 208) based on an analysis of analyzing the frame array. In a further example, the analyte quantity can be determined by applying a trained machine learning model (e.g., an ANN or CNN) to analyze elements of the frame array for the at least some (e.g., a subset) of the respective image frames to determine the quantity of the at least one analyte in the sample. In another example, the machine learning model is applied to each of the elements of the frame array to determine the quantity of the at least one analyte in the sample.

At 210, the method can also include analyzing the at least some of the image frames to determine at least one analyte variant for the at least one analyte identified in the sample. In an example when the analyte includes hemoglobin, the method further comprising analyzing the pixel values of the respective image frames to identify at least one hemoglobin variant for the sample (e.g., HbAA, HbSA, HbSS, HbSC, and/or HbA2).

In a further example, analysis performed at 210 for identifying one or more analyte variants can include applying a trained machine learning model to analyze at least some of the respective image frames to determine the variants of the at least one analyte in the sample. For example, the machine learning model can be implemented as a CNN trained to extract and classify features and to predict the identity and percentage (e.g., a fractional part) for each of the analyte variants. The features can be extracted from the image frames. Additionally, or alternatively, the features can be extracted from a frame array that has been generated to include elements encoding of information in each of the respective image frames for some or all of the time interval. That is, each element of the array can encode corresponding image information in a respective image frame. The information encoded can include spatial and temporal information for the electrophoresis process, such as pixel values (e.g., scaled pixel values) for one or more color channels during the electrophoresis process. In a high resolution version, each element can include an entire ROI of the image frame. Thus, the extent of the information can range from a single value derived to encode an image frame up to including the entire image (or a derivative thereof). In any such example, regardless of the extent of information included in the frame array, the trained machine learning model (e.g., CNN) can be applied to analyze the elements of the frame array to determine the variants of the at least one analyte in the sample. The frame array analyzed at 210 can be the same frame array used in the method at 208 to estimate the analyte quantity, or, alternatively, the frame array used at 210 can be generated as a different frame array (e.g., containing more information and/or more dimensions of information) than the frame array used at 208.

The method 200 further can include generating a diagnostic output based on the quantity and/or identity of one or more analytes. The diagnostic output can be provided to a display or other output device. Additionally, or alternatively, the diagnostic output can be communicated from the diagnostic device (e.g., a portable device) to a remote system, such as through a communication link.

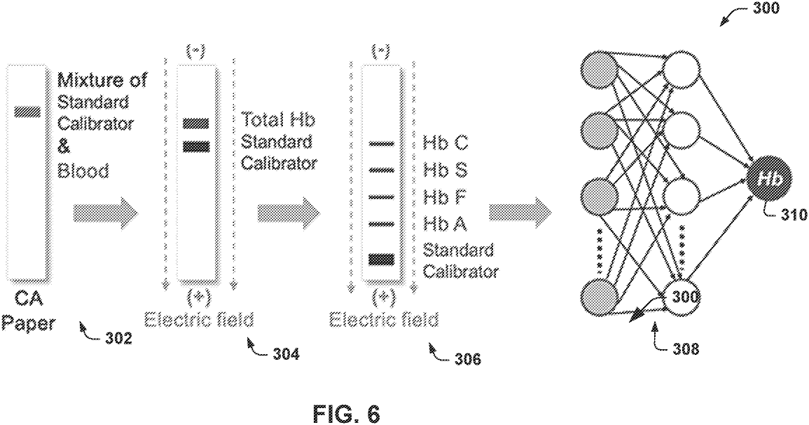

In view of the foregoing systems and methods of FIGS. 1-5, the concept of analyzing images of an electrophoresis process applied to a blood sample will be further appreciated with respect to FIGS. 6-10C.

FIG. 6 depicts a schematic representation of an example diagnostic process, shown at 300. For example, a sample of Blood is mixed with a calibrator (e.g., xylene cyanol) and applied on an electrophoresis medium (e.g., paper or gel medium), such as shown at 302. The medium can be in a cartridge that is coupled to a receptacle of an electrophoresis system (e.g., system 102). The electrophoresis process can be initiated in response to the cartridge being installed or a user input. Within a first portion of the test interval (e.g., t ≤ 2.5 min) the total hemoglobin analyte and standard calibrator are electrophoretically separated, shown at 304. Images can be acquired during the electrophoresis process, including images of the electrophoretic separation that occurs over time, to provide image frames. During a second portion of the test interval hemoglobin variant separation occurs (e.g., 2.5 min ≤ t ≤ 8 min). For example, the image frames acquired during the electrophoresis process can be analyzed, as disclosed herein to determine the hemoglobin level (e.g. in g/dL) as well as to determine the presence of hemoglobin variants and types in the blood sample (i.e., Hb A, F, S, and C).

As described herein, the entire electrophoresis process can be tracked in real-time by computer vision to provide image data (e.g., image data 110) representing an image frames visualizing of the electrophoresis test medium over time during application of an electric field across the test medium containing a sample under test (e.g., blood). As shown in the example schematic illustration of FIG. 6, one or more deep learning artificial neural network (ANN) algorithm 308 can be trained to analyze the image data to perform integrated blood Hb level prediction (e.g., shown at 310), anemia detection, and Hb variant identification in a single test device (e.g., diagnostic system 100).

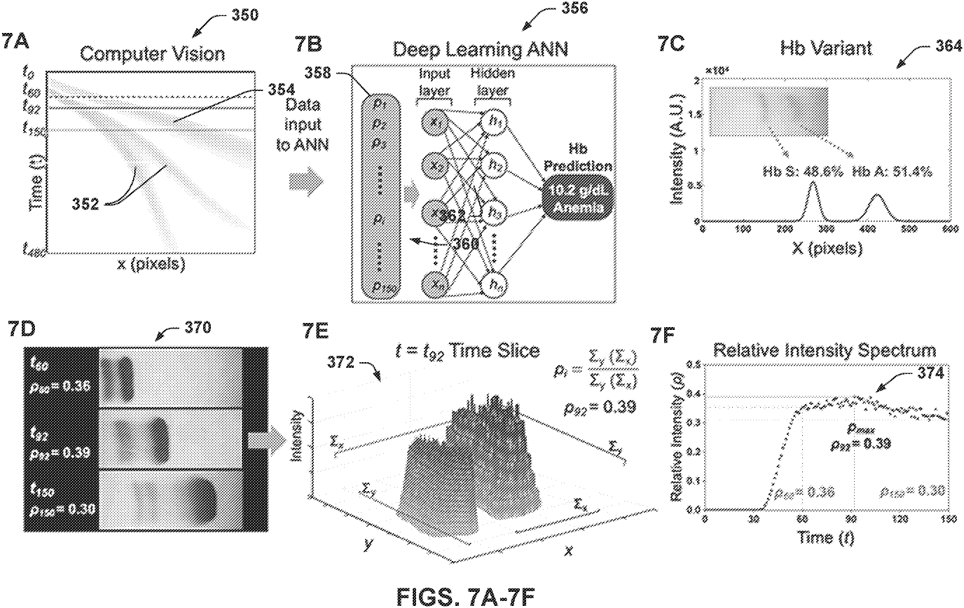

FIGS. 7A-7F illustrate an example overview of an integrated system workflow that can be employed to perform hemoglobin level prediction, anemia detection, and hemoglobin variant identification. The workflow can be implemented according to the systems and methods of FIGS. 1-5. FIG. 7A shows an example two-dimensional representation of a time-space plot 350 visually illustrating the full electrophoretic band separation process in a single image. For example, the plot shows trajectory of a blood analyte 352 and calibrator 354 over time (y-axis) and space (x-axis) based on image data acquired for an electrophoresis process. In FIG. 7A, each pixel row of the image corresponds to a single one frame, with time increasing from top (0 s) to bottom (480 s). For each point on the x-axis, the plot includes the total intensities for the two-color bands (e.g., red = Hb and blue = standard calibrator), summed across the y-axis in the range of the region of interest (ROI). The ROI can be illustrated in the inset for a representative video frame.

FIG. 7B schematically illustrates the process in which information (e.g., band information) in respective image frames is analyzed to generate a time series vector ρ(t)for t=0 to 150, shown at 358. In another example, the time series vector p(t) can be generated for the entire time interval (e.g., t=0 to 480 s) For example, the time series vector p(t) can be generated by array generator (e.g., generator 164 or 182) based on evaluating the relative intensity between Hb band 352 and the standard calibrator band 354 over the time interval. The time series vector p(t) can be provided as an input 358 for the trained ANN, shown at 360. The ANN 360 thus can be programmed (e.g., corresponding to model 170) to predict the Hb level based on an analysis of the input 358, as shown at 362. A corresponding anemia diagnosis (e.g., a binary diagnosis indicating positive or negative for anemia) can be provided based on the Hb level.

FIG. 7C shows an example one or more Hb variants being identified (e.g., by variant calculator 134). For example, the Hb variants can be identified based on the final location Hb variant band at or near the end of the test interval (e.g., t=480 s).

FIG. 7D illustrates an example of respective image frames 370 within the ROI at 3 representative time points acquired during the electrophoresis test process. For example, at 60 s, detectable separation initiated between Hb band and standard calibrator band due to their major mobility differences while the Hb band remain unseparated (Top frame, showing relative intensity of ρ₆₀= 0.36).At 92 s, Hb band and standard calibrator band further separate thus increasing band separation resolution (Middle frame, showing relative intensity of ρ₉₂ = 0.39). The relative intensity values for each frame can be determined by the relative intensity calculator 162, such as described herein. At 150 s, total hemoglobin starts to separate into respective hemoglobin variants due to their minor mobility differences (Bottom frame, showing relative intensity of ρ₁₅₀= 0.30).

FIG. 7E shows an example 3D intensity profile 372 extracted from image acquired at t = 92 s (e.g., shown at solid horizontal line 373 shown in FIG. 7A and middle image frame in FIG. 7C). For example, the relative intensity for a given frame can be calculated (e.g., by relative intensity calculator 162) as a spatial summed relative intensity between red channel and blue channels (ρᵢ = (∑_{y} ∑ₓ )_{Red} /(∑_{y}∑ₓ )_{Blue}. FIG. 7F shows an example pattern of time series vector ρ(t) including ρ₁ to ρ₁₅₀ recognized by the trained ANN (e.g., ML model 170).

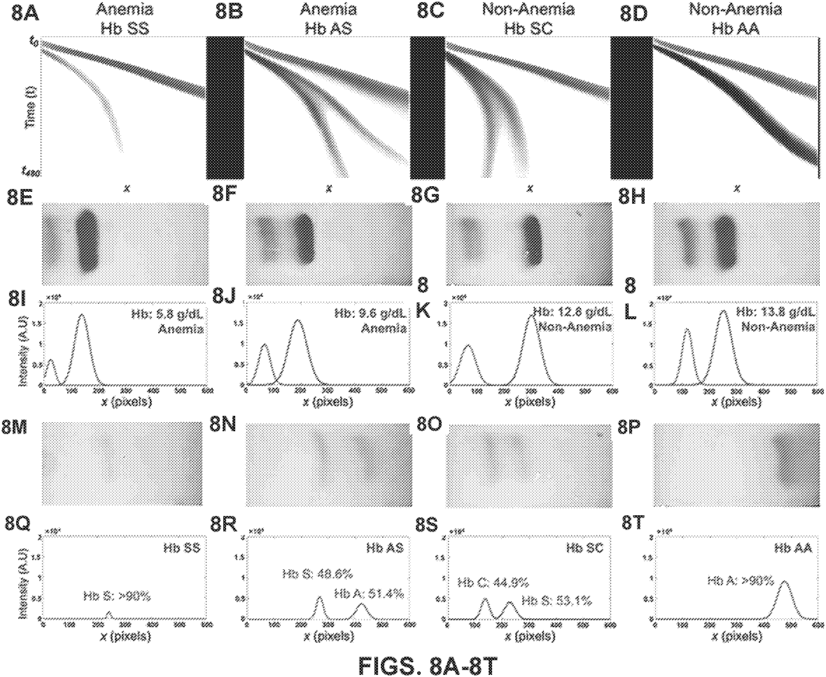

FIGS. 8A-8T illustrate further examples of integrated Hb level prediction, anemia detection and Hb variant identification in 4 representative Hemoglobin Variant/Anemia electrophoresis tests performed on clinical blood samples at different Hb levels and Hb variant phenotypes. For example, FIGS. 8A-8D (e.g., the first horizontal row of FIGS. 8A-8T) includes 2D representations of HbV A test band trajectories, such as can be derived from image frames acquired during electrophoresis tests performed (e.g., by electrophoresis system 102) on the respective samples.

FIGS. 8E-8H (e.g., the second horizontal row of FIGS. 8A-8T) illustrate a representative frame for each test from the image frames, such as can be used (e.g., by analyte quantity calculator 132) to generate relative intensity ratio time series vectors ρ(t) (or another frame array) to encode the information in the respective image frames. The time series vectors ρ(t)(or another frame array) can then utilized by the ANN (e.g., ML model 170) to predict the Hb levels, such as disclosed herein.

FIGS. 8I-8L (e.g., the third horizontal row of FIGS. 8A-8T) demonstrate the electropherogram corresponding to the image frames in the second row generated from the intensity profile envelopes, thus including spatial and temporal information derived from the respective image frames. The predicted Hb levels can be compared against a reference method, such as complete blood count (CBC) reported results.

FIGS. 8M-8P (e.g., the fourth horizontal row of FIGS. 8A-8T) demonstrate examples of image frames that can be utilized (e.g., by analyte variant calculator 134) to identify Hb variants. For example, the Hb variants can be determined using another ML model or by another algorithm, such as disclosed herein. FIGS. 8Q-8T (e.g., the fifth horizontal row of FIGS. 8A-8T) demonstrate examples of electropherograms generated according to the band information in the fourth row (FIGS. 8M-8P).

In FIGS. 8A-8T, each column represents respective test data and results data for each sample (e.g., obtained from different patients). For example, first column: HbVA test result for patient at Hb level of 6.0 g/dL and with homozygous HbSS (sickle cell disease, SCD patient); Second column: HbV A test result for patient at Hb level of 10.3 g/dL and with heterozygous HbAS (SCD patient undergoing transfusion therapy); Third column: HbVA test result for patient at Hb level of 12.7 g/dL and with heterozygous Hb SC disease (hemoglobin C disease); Fourth column: HbVA test results for patient at Hb level of 14.5 g/dL and with homozygous HbAA (healthy subject).

The HbVA Hb level prediction and anemia detection results are compared against the reference method complete blood count (CBC) reported results. The Hb variant identified by HbVA are compared against the reference method high performance liquid chromatography (HPLC) reported results. HbVA demonstrated agreement in Hb level prediction, anemia detection and Hb variant identification with reference standard methods CBC and HPLC. (Patient 1: HbVA: 5.8 g/dL, Anemia, Hb SS vs. CBC&HPLC: 6.0 g/dL, Anemia, Hb SS; Patient 2: HbVA: 9.6 g/dL, Anemia, Hb AS vs. CBC&HPLC: 10.3 g/dL, Anemia, Hb AS; Patient 3: HbVA: 12.8 g/dL, Anemia, Hb SC vs. CBC&HPLC: 12.7 g/dL, Anemia, Hb SC; Patient 4: HbVA: 13.8 g/dL, Non-anemia, Hb AA vs. CBC&HPLC: 14.5 g/dL, Non-anemia, Hb AA). These results demonstrate the capability of enabling integrated blood Hb level prediction and Hb variant identification using systems and methods disclosed herein.

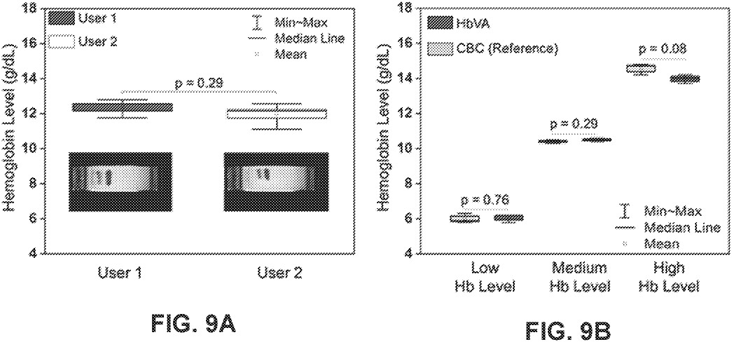

FIGS. 9A-9B includes plots to illustrate robustness and reproducibility of the systems and methods disclosed herein for determining hemoglobin variant/anemia (HbVA) Hb level prediction. For example, FIG. 9A is a plot showing results for an example robustness test of HbVA Hb level prediction, which was tested using the systems and methods herein with 10 repeated tests using the same sample, comparing variances between 2 users (demonstrated in the inset figures). As shown in the example of FIG. 9A, no significant difference (p=0.29) was observed between Hb level predicted from user 1 HbVA tests (filled red round, Mean ± Standard Deviation = 12.3±0.4 g/dL, n=5, left inset) and Hb levels predicted from user 2 HbVA tests (opened red circle, Mean ± Standard Deviation = 12.0±0.6 g/dL, n=5, right inset). Hb level predicted by all 10 repeated tests demonstrated agreement of ±1.0 g/dL against the 12.7 g/dL Hb level reported by reference standard of complete blood count (CBC).

FIG. 9B is a plot showing results for an example reproducibility test of HbVA Hb level prediction, which was tested using 3 samples with low, middle and high Hb levels reported from CBC. Each sample was tested 3 times by both HbVA and CBC. The standard deviation of HbVA predicted Hb levels are within 4% CV across low, middle and high Hb levels (Low Hb level: HbVA: Mean ± Standard Deviation = 6.1±0.2 g/dL, CV% = 3.8% Middle Hb level: Mean ± Standard Deviation = 10.5±0.1 g/dL, CV% = 1.0% and High Hb level Mean ± Standard Deviation = 14.0±0.3 g/dL, CV% = 2.1%). The HbVA predicted Hb levels also agree with the reference standard CBC reported Hb levels (Low Hb level: Mean ± Standard Deviation = 6.0±0.3 g/dL, CV% = 5.0%; Middle Hb level: Mean ± Standard Deviation = 10.4±0.1 g/dL, CV% = 1.0%; High Hb level: Mean ± Standard Deviation = 14.6±0.3 g/dL, CV% = 2.1%). HbVA predicted Hb levels from all 3 groups of tests were within ±0.6 g/dL and ±5.0% with the CBC reported. No significant difference was found between the Hb level measured using HBVA against reference method CBC through tested Hb range (p=0.76, 0.29 and 0.08, respectively). n=3 for each test.

FIGS. 10A-10C illustrate plots demonstrate that the system and methods disclosed herein (e.g., HbVA ANN based deep learning algorithm) accurately predicts Hb levels. FIG. 10A is an example plot showing blood Hb levels (e.g., determined by HbVA ANN) are strongly associated with CBC measured results (PCC=0.95, p<0.001). The dashed line represents the ideal result where HbVA Hb level is equal to the CBC Hb level, whereas solid line represents the actual data fit. FIG. 10B is a plot of Bland-Altman analysis, which reveals an example HbVA predicts blood Hb levels to within ±0.55 of the Hb level (absolute mean error) with minimal experimental bias with -0.1 g/dL, indicating that Hb prediction has very small bias. The dashed light grey line indicates the relationship between the residual and the average Hb level measurements obtained from the CBC and HBVA (r = -0.07). The dashed dark grey line represents 95% limits of agreement (±1.5 g/dL). FIG. 10B is a plot of the receiver-operating characteristic (ROC) analysis, which graphically illustrates example HbVA’s performance against a random chance diagnosis (grey line), with an area under the curve of 0.99, and a perfect diagnostic (green lines), with an area under the curve of 1. The area under the curve of 0.99 suggests HbVA’s viable diagnostic performance. n=46.

In view of the foregoing, examples of systems and methods disclosed herein can employ computer vision and deep machine learning to extract previously inaccessible new information from the electrophoresis, such as for enabling, for the first time, reproducible, accurate, and integrated blood analyte level prediction, diagnostic detection, and analyte variant identification, which can be implemented in a single integrated point-of-care test. Additionally, the testing systems and methods disclosed herein can be used by minimally trained personnel to produce fast, accurate, and reproducible results. The systems and methods thus can be used to meet increasing needs in biology, medicine and chemistry, such as when rapid, decentralized sample analysis is needed.

In view of the foregoing structural and functional description, those skilled in the art will appreciate that portions of the systems and method disclosed herein may be embodied as a method, data processing system, or computer program product such as a non-transitory computer readable medium. Accordingly, these portions of the approach disclosed herein may take the form of an entirely hardware embodiment, an entirely software embodiment (e.g., in a non-transitory machine readable medium), or an embodiment combining software and hardware. Furthermore, portions of the systems and method disclosed herein may be a computer program product on a computer-usable storage medium having computer readable program code on the medium. Any suitable computer-readable medium may be utilized including, but not limited to, static and dynamic storage devices, hard disks, flash drives optical storage devices, and magnetic storage devices.

Certain embodiments have also been described herein with reference to block illustrations of methods, systems, and computer program products. It will be understood that blocks of the illustrations, and combinations of blocks in the illustrations, can be implemented by computer-executable instructions. These computer-executable instructions may be provided to one or more processor of a general purpose computer, special purpose computer, or other programmable data processing apparatus (or a combination of devices and circuits) to produce a machine, such that the instructions, which execute via the processor, implement the functions specified in the block or blocks.

These computer-executable instructions may also be stored in computer-readable memory that can direct a computer or other programmable data processing apparatus to function in a particular manner, such that the instructions stored in the computer-readable memory result in an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions described herein.

What have been described above are examples. It is, of course, not possible to describe every conceivable combination of structures, components, or methods, but one of ordinary skill in the art will recognize that many further combinations and permutations are possible. Accordingly, the invention is intended to embrace all such alterations, modifications, and variations that fall within the scope of this application, including the appended claims. Where the disclosure or claims recite “a,” “an,” “a first,” or “another” element, or the equivalent thereof, it should be interpreted to include one or more than one such element, neither requiring nor excluding two or more such elements. As used herein, the term “includes” means includes but not limited to, and the term “including” means including but not limited to. The term “based on” means based at least in part on.

We claim:

1. A system comprising:
one or more non-transitory machine readable media configured to store data and instructions, the data comprising image data, the image data including a plurality of image frames of an electrophoresis process performed on a sample over a time interval, the sample containing at least one analyte;
one or more processors configured to access the data and execute the instructions, the instructions programmed to perform a method comprising:
determining values of pixels within a region of interest (ROI) of respective image frames in the time interval;
analyzing the determined pixel values for at least one of the respective image frames; and
estimating a quantity of the at least one analyte in the sample based on the analysis of the pixel values.

2. The system of claim 1, wherein the method further comprises:
generating a frame array having elements representative of information in respective image frames for at least a portion of the time interval; and
analyzing the elements in the frame array to estimate the quantity of the at least one analyte in the sample.

3. The system of claim 2, wherein the respective image frames are multi-channel images, in which a first color channel represents values of pixels corresponding to the at least one analyte in the sample or an absence of the at least one analyte from the sample, and a second color channel represents values of pixels corresponding to a calibrator in the sample,
wherein generating the frame array further comprises:
determining a relative pixel value for each of the image frames based on values of pixels in the first color channel and values of pixels in the second color channel for the respective image frame; and
storing the relative pixel values to define respective elements of the frame array, so that the frame array comprises a vector of the relative pixel values over the time interval.

4. The system of claim 3, wherein the method further comprises:
scaling values of respective pixels for each color channel based on a threshold operator to provide scaled values for pixels in each color channel of each of the respective image frames;
wherein the relative pixel value for each of the image frames is determined based on the scaled values for pixels of each of the respective image frames.

5. The system of claim 2, wherein analyzing the frame array comprises:
applying a trained machine learning model to elements of the frame array for the at least some of the respective image frames to determine the quantity of the at least one analyte in the sample.

6. The system of claim 5, wherein the machine learning model is applied to the elements of the frame array for each of the respective image frames in the time interval to determine the quantity of the at least one analyte in the sample.

7. The system of claim 1, wherein the sample is a blood sample and the quantity of the at least one analyte comprises an indication of blood hemoglobin level in the sample, wherein the method further comprises providing a diagnosis of anemia based on the indication of blood hemoglobin level in the sample.

8. (canceled)

9. The system of claim 1, wherein the sample is a blood sample and the quantity of the at least one analyte comprises a serum protein level in the sample.

10. The system of claim 1, wherein the method further comprises
analyzing the pixel values to determine at least one analyte variant for the at least one analyte identified in the sample.

11. The system of claim 10, wherein the at least one analyte variant includes hemoglobin, the method further comprising analyzing the pixel values of the respective image frames to identify at least one hemoglobin variant for the sample.

12. The system of claim 11, wherein the at least one hemoglobin variant is a hemoglobin phenotype comprises at least one of HbAA, HbSA, HbSS, HbSC, and HbA2.

13. The system of claim 10, wherein analyzing the pixel values to determine at least one variant of the at least one analyte further comprises applying a trained machine learning model to analyze at least some of the respective image frames to determine the at least one variant of the at least one analyte in the sample.

14. The system of claim 1, wherein the respective image frames are multi-channel images having multiple color channels, wherein determining values of pixels further comprises:
scaling values of pixels for a respective color channel with respect to other color channels in each of the respective image frames to provide scaled values for the pixels in each the respective image frames, wherein scaling values of pixels further comprises:
normalizing a value of each pixel in a first color channel, which represent pixels corresponding to the at least one analyte in the sample, based on values of each respective pixel in other color channels, and
normalizing a value of each pixel in a second color channel, which represent pixels corresponding to a calibrator in the sample, based on values of each respective pixel in other color channels.

15. (canceled)

16. The system of claim 14, wherein the method further comprises:
determining a relative intensity of pixels in each of the respective image frames based on the values of respective pixels in the first color channel and values of respective pixels in the second color channel; and
generating a time-series vector that includes the relative intensity of pixels determined for the respective image frames, wherein the quantity of the at least one analyte in the sample is estimated based on the time series vector.

17. The system of claim 16, wherein estimating the quantity of the at least one analyte in the sample comprises:
applying a trained machine learning model to the time-series vector for the at least some of the respective image frames to determine the quantity of the at least one analyte in the sample.

18. The system of claim 1, the method further comprising identifying at least one analyte variant based on analysis of spatial and temporal features of pixels in at least one image frame at or near an end of the time interval.

19. The system of claim 1, further comprising:
an electrophoresis system that includes an electrophoresis medium configured to hold the sample, a visual property of the electrophoresis medium varying in response to the electrophoresis process based on a charge and mass of the at least one analyte in the sample; and
an imaging system configured to acquire images of the electrophoresis medium at a frame rate during the time interval to provide the image data.

20. The system of claim 19, further comprising a portable device having a housing that includes the one or more machine readable media, the one or more processors and the electrophoresis system.

21. The system of claim 19, further comprising a display, the method further comprising generating a diagnostic output based on the quantity of the at least one analyte and providing the diagnostic output to the display.

22. A method comprising:
storing image data that includes image frames of an electrophoresis process performed on a sample over a time interval;
determining values of pixels within a region of interest (ROI) of respective image frames in the time interval;
analyzing the pixel values for at least some of the respective image frames; and
estimating a quantity of at least one analyte in the sample based on the analysis of the pixel values.

23. The method of claim 22, wherein the at least one of the respective image frames includes a plurality of image frames in the time interval, and the method further comprises:
generating a frame array having elements that encode information in the respective image frames for at least a portion of the time interval; and
analyzing the frame array to estimate the quantity of the at least one analyte in the sample..

24. (canceled)

25. The method of claim 23, wherein the respective image frames are multi-channel images, in which a first color channel represents values of pixels corresponding to the at least one analyte in the sample or an absence of the at least one analyte from the sample, and a second color channel represents values of pixels corresponding to a calibrator in the sample,
wherein generating the frame array further comprises:
determining a relative pixel value for each of the image frames based on values of pixels in the first color channel and values of pixels in the second color channel for the respective image frame; and
storing the relative pixel values to define respective elements of the frame array, so that the frame array comprises a vector representing the relative pixel values for one or more color channels over the time interval.

26. The method of claim 25, further comprising:
scaling values of respective pixels for each color channel to provide scaled values for pixels in each color channel of each of the respective image frames;
wherein the relative pixel value for each of the image frames is determined based on the scaled values for pixels of each of the respective image frames.

27. The method of claim 24, wherein analyzing the frame array comprises:
applying a trained machine learning model to analyze elements of the frame array for the at least some of the respective image frames to predict the quantity of the at least one analyte in the sample, wherein the machine learning model is applied to each of the elements of the frame array to determine the quantity of the at least one analyte in the sample.

28. (canceled)

29. The method of claim 22, wherein the sample is a blood sample and the quantity of the at least one analyte comprises an indication of blood hemoglobin level in the sample, wherein the method further comprises providing a diagnosis of anemia based on the indication of blood hemoglobin level in the sample.

30. (canceled)

31. The method of claim 22, wherein the sample is a blood sample and the quantity of the at least one analyte comprises an indication of serum protein level in the sample.

32. The method of claim 22, wherein the method further comprises
analyzing the at least some of the image frames to determine at least one analyte variant for the at least one analyte identified in the sample.

33. The method of claim 32, wherein the at least one analyte includes hemoglobin, the method further comprising analyzing the pixel values of the respective image frames to identify at least one hemoglobin variant for the sample.

34. The method of claim 33, wherein analyzing the pixel values to determine variants of the at least one analyte further comprises applying a trained machine learning model to analyze at least some of the respective image frames to determine the variants of the at least one analyte in the sample.

35. The method of claim 34, further comprising:
generating a time-based frame array having elements that encode of information in each of the respective image frames for at least a portion of the time interval; and
applying the trained machine learning model to analyze the elements of the frame array to determine the variants of the at least one analyte in the sample.

36. The method of claim 22, wherein the respective image frames are multi-channel images having multiple color channels, and wherein determining values of pixels further comprises:
scaling value of each pixel for a respective color channel with respect to values of the pixel for other color channels in each of the respective image frames to provide scaled values for the pixels in each the respective image frames, wherein
scaling values of pixels further comprises:
normalizing a value of each pixel in a first color channel, which represent pixels corresponding to the at least one analyte in the sample, based on values of each respective pixel in other color channels, and
normalizing a value of each pixel in a second color channel, which represent pixels corresponding to a calibrator in the sample, based on values of each respective pixel in other color channels.

37. (canceled)

38. The method of claim 36, further comprising:
determining a relative intensity of pixels in each of the respective image frames based on the values of respective pixels in the first color channel and values of respective pixels in the second color channel; and
generating a time-series vector that includes the relative intensity of pixels determined for the respective image frames, wherein the quantity of the at least one analyte in the sample is estimated based on the time series vector.

39. The method of claim 38, wherein estimating the quantity of the at least one analyte in the sample comprises:
applying a trained machine learning model to analyze the time-series vector for the at least some of the respective image frames to determine the quantity of the at least one analyte in the sample; and
identifying at least one analyte variant for the at least one analyte in the sample based on analysis of spatial and temporal features of pixels in at least one of the image frames.

40. (canceled)

41. The method of claim 22, further comprising:
holding the sample in an electrophoresis medium;
generating an electric field across the electrophoresis medium so a visual property of the electrophoresis medium varies in response to the electric field based on a charge and mass of the at least one analyte in the sample;
acquiring images of the electrophoresis medium at a frame rate during the time interval to provide the image data; and
generating a diagnostic output based on the quantity of the at least one analyte and providing the diagnostic output to a display.

42. (canceled)

43. A system comprising:
an electrophoresis system that includes an electrophoresis medium configured to hold a blood sample containing at least one blood analyte and a known calibrator;
an imaging system configured to acquire an image of the electrophoresis medium at a frame rate to provide image data having at least one image frame representative of an electrophoresis process performed on the sample over a time interval;
one or more non-transitory machine readable media configured to store data and instructions, the data comprising the image data; and
one or more processors configured to access the data and execute the instructions, the instructions comprising:
an analyte quantity calculator programmed to at least:
generate encoded image data to encode image information within a region of interest (ROI) of at least one respective image frame of a plurality of frames acquired during the electrophoresis process, and
apply a machine learning model to analyze the encoded image data and provide an indication of a property of the at least one blood analyte in the sample.

44-50. (canceled)
